(19) United States
(12) Patent Application Publication
(10) Pub. No.: US 2007/0166295 A1
(43) Pub. Date: Jul. 19, 2007

(54) Anti-Diarrhea Preparations for Small Domestic Animals

(75) Inventors: Daniel J. Schildgen, Colleyville, TX (US); Heather Fox, Bedford, TX (US)

Correspondence Address: MCGARRY BAIR PC, 32 Market Ave. SW, SUITE 500, GRAND RAPIDS, MI, 49503, US

(73) Assignee: VET SOLUTIONS L.P., Fort Worth, TX (US)

(21) Appl. No.: 11/557,766

(22) Filed: Nov. 8, 2006

Related U.S. Application Data
(60) Provisional application No. 60/743,127, filed on Jan. 13, 2006.

(51) Int. Cl. A61K 35/74; A61K 33/06; A61K 31/732

(52) U.S. Cl. 424/93.45; 424/684; 514/54

(57) ABSTRACT

A composition and method for treating diarrhea in small domestic animals wherein the composition includes an effective amount of kaolin, pectin, at least one probiotic, and a carrier, and the method includes the step of orally administering an effective dosage of the composition to the domestic animals.

### CROSS-REFERENCE TO RELATED APPLICATIONS

This application claims the benefit of U.S. Provisional Patent Application Ser. No. 60/743,127, filed Jan. 13, 2006, which is incorporated herein by reference.

### BACKGROUND OF THE INVENTION

1. Field of the Invention

This invention relates to anti-diarrhea preparations for small domestic animals such as dogs and cats. In one of its aspects, the invention relates to anti-diarrhea gel preparation for oral ingestion by small domestic animals. In another of it aspects, the invention relates to anti-diarrhea preparations in the form of a tablet with flavoring for oral ingestion by small domestic animals. In another aspect, the invention relates to a method of treating, preventing, or reducing the risk of diarrhea in small domestic animals such as dogs and cats.

2. Description of the Related Art

Diarrhea, or loose stool, is a very common problem in pets and occurs for a variety of reasons. Some common causes of mild, abrupt onset diarrhea include change in diet, feeding of table scraps, intestinal parasites, and antibiotic administration and stress associated with boarding, travel, or surgery. When a pet experiences diarrhea, the normal balance of fluids and solids in the intestines is upset, the intestinal lining may be irritated, and the population of normal bacteria that protect against diseases may be reduced or changed. In many cases, the diarrhea is not life threatening but needs appropriate treatment.

Many anti-diarrhea preparations are available for animals as well as humans. A variety of different compounds and compositions have been proposed. Many of these preparations are designed for humans and a number of preparations have been designed especially for domestic animals. Others have been developed especially for large animals such as horses and farm animals. Many of these preparations are packaged in the form of gel and are directly fed in an oral administration through a syringe. Some of the preparations include beneficial lactic acid-producing bacteria including Lactobacillus Acidophilus and Enterococcus Faecium. These bacteria are known as “probiotic bacteria”. One particular preparation for dogs is marketed under the trademark Probios® and is in the form of tablets that include Lactic Acid Bacteria along with Inulin. The bacteria can include Enterococcus faecium, Lactobacillus acidophilus, Lactobacillus plantarum, and Lactobacillus casei. This product is manufactured and sold by Vets Plus, Inc.

Another veterinary preparation for animals is sold under the mark Fast Balance—G.I. by Vetri-Science Laboratories of Vermont. Fast Balance—G.I. preparation comprises pantothenic acid, vitamin B6, vitamin B2, mixed lactic acid bacteria, Saccharomyces cerevisiae, Bacillus subtilis, and Aspergillus oryzae, along with a number of inactive ingredients. Still another probiotic preparation is sold by Provita Eurotech Limited of Omagh, Co., Tyrone, Northern Ireland, under the mark Provita for cats and kittens and for dogs and puppies. The preparation comprises a probiotic paste.

The U.S. Patent Appliclation Publication No. 2003/0143293 to Shushunov, published Jul. 31, 2003, discloses a composition comprising an anti-diarrheal agent and an electrolyte that can be used for veterinary treatment of animals. The anti-diarrheal agent preferably is tormentil root extract, but can include a wide variety of compounds including Kaolin-Pectin. In the Shushunov compositions electrolytes are compounds that conduct electricity while in solution and are decomposed (electrolyze) by the solution. This Shushunov composition can be used in combination therapy with various other therapeutic agents that include probiotics, immunoglobulins, antiperistaltic and antisecretory agents, such as different preparations of tannins, bismuth subsalicylate, antibiotics and the like.

### SUMMARY OF THE INVENTION

According to the invention, a composition for treating diarrhea in small domestic animals, such as dogs and cats, comprises effective amounts of kaolin, pectin, and at least one probiotic, with a carrier. The composition can be in the form of a gel or a tablet. As used herein, the term “effective amounts” or “effective amount” means that amount of each ingredient, singularly, or in combination, that renders the composition effective in treating diarrhea in small domestic animals such as dogs and cats.

The ratio of kaolin to pectin is significant for this composition and typically is in the range of about 35:1 to 15:1 and preferably about 25:1.

The probiotic can be any one of a number of beneficial bacteria that include any one of the following:

Aspergillus niger;Aspergillus oryzae; Bacillus lentus; Bacillus licheniformis; Bacillus puilus; Bacillus subtilis; Bacteroides amylophilus; Bacteroides capillosus; Bacteroides ruminocola; Bacteroides suis; Bifidobacterium adolescentis; Bifidobacterium animalis; Bifidobacterium bifidum; Bifidobacterium infantis; Bifidobacterium longum; Bifidobacterium thermophilum; Lactobacillus acidophilus; Lactobacillus brevis; Lactobacillus buchneri; Lactobacillus bulgaricus; Lactobacillus casei; Lactobacillus cellobiosus; Lactobacillus curvatus; Lactobacillus delbruekii; Lactobacillusfermentum; Lactobacillus helveticus; Lactobacillus lactis; Lactobacillus plantarum; Lactobacillus euterii (probably reuterii or reuteri); Leuconostoc mesenteroides; Pediococcus acidilacticii; Pediococcus cerevisiae (damnosus); Propionibacterium freudenreichii; Propionibacterium shermanii; Saccharomyces cerevisiae; Enterococcus cremoris; Enterococcus diacetylactis; Enterococcusfaecium (E. faecium); Enterococcus intermedius; Enterococcus lactis; Enterococcus thermophilus; Yeast.

Preferably, the probiotic is Enteroccoccus faecium (E. facecium) which has been freeze dried and microencapsulated.

In the gel form of the composition, soy oil is the carrier. In the tablet form, the carrier can be any one of a number of carriers, which include sorbitol and maltodextrin. In one embodiment of invention, the carrier includes sorbitol in the range of 10-40%, preferably in the range of 26-32%. In another embodiment of the invention, the carrier includes maltodextrin in the range of 5-25%, preferably in the range of 12-15%. Preferably, the sorbitol and the maltodextrin combine to form the carrier.

Further, the composition can include flavoring that is attractive to domestic animals. In the preferred embodiment of the invention, the flavoring is liver but the flavoring can be any suitable common flavoring that is attractive to animals.

Still further according to the invention, a method of treating diarrhea in small domestic animals such as dogs and cats comprises orally administering an effective dosage of the compositions according to the invention as set forth above to the animal. In one embodiment, the method includes orally administering the dosage by injecting the dosage in gel form on the animal's tongue with a syringe. In another embodiment of the invention, the method includes orally administering an effective dosage of the composition to the animal by mixing the effective dosage of the composition with the animal's food or otherwise feeding the tablets directly to the animal.

### DESCRIPTION OF THE PREFERRED EMBODIMENT

Kaolin is fine, usually white, clay that is used in medicine, especially as an adsorbent in the treatment of diarrhea. Typically, kaolin soothes the irritated intestines, adsorbs water, and adsorbs any excess bacterial toxins which may be produced when a pet has diarrhea. By absorbing water, kaolin helps to form stool and helps the pet feel better. When the composition is in gel form, kaolin is present in amounts of about 20-80 parts by weight, preferably in the range of about 47-50% by weight. When in tablet form, the kaolin is present in amounts of about 10-50% parts by weight, preferably about 20-30% parts by weight and mostly present in about 25% parts by weight.

Pectin is a form of fiber found in fruit and helps to absorb water and firm stool. Pectin is present in the composition when in gel form generally in a range of about 4.5-10% weight, and preferably in the range of about 1.9-2.0% by weight. When in tablet form, Pectin is present in the composition in the range of 0.5% to 5% by weight, and preferably in the range of 1% to 2% by weight, and most preferably about 1.25% by weight.

E. faecium is a part of the normal intestinal flora. Microencapsulation helps the bacteria survive in the very acid environment of the stomach and move into the intestines. Once in the intestines, the bacteria will help restore the population of normal bacteria and eliminate “bad” bacteria by competing with bad bacteria for space and nutrients. Eliminating bad bacteria and restoring the population of good bacteria will help to restore normal intestinal function and eliminate diarrhea.

The amount of probiotics in the composition can range over a fairly wide spectrum. Generally, the amount of probiotics in the composition will be about 50-400 million colony-forming units per gram of the composition, preferably in the range of 100-200 million colony-forming units per gram.

The carrier can comprise a number of well known carriers for gels and for tablets. For example, in the gel form of the composition, the carrier can be any suitable edible oil, including soy oil, olive oil, corn oil, sunflower oil, safflower oil, canola oil, peanut oil, and any other vegetable oil. The oil can be present in the gel form in the amount of 20-60% by weight, preferably in the range of the 41-44 weight percent.

In the tablet form, the carrier can be any one of a number of carriers, which include sorbitol and maltodextrin. The carrier can be present in the tablet composition in the range of 5 to 40%, preferably in the range of 12 to 35% by weight. When Sorbitol is used in the composition, it can be present in the range of 10-40%, preferably in the range of 26-32%. When Maltodextrin is used in the composition, it can be present in the range of 5-25%, preferably in the range of 12-15%. Preferably, the sorbitol and the maltodextrin combine to form the carrier.

### SPECIFIC EXAMPLES

### Example 1

A gel composition according to the invention was made with the following ingredients:

| | GRAMS PER MILLILITER |
|---|---|
| | (EXCEPT AS OTHERWISE |
| INGREDIENT | NOTED) |
| Kaolin | 0.6 |
| soy oil | 0.526 |
| Pectin | 0.025 |
| Encapsulated dried | 0.2 billion colony-forming units |
| Enterococcus faecium | (0.157 billion units per gram) |
| Fermentation product | |
| Powered sugar | 0.0084 |
| Chicken flavor | 0.03 |
| artificial and natural flavors | 0.018 |

1.2074 gm total per milliliter

The gel is loaded into 15cc and 30cc dial-a-dose syringes for oral administration of the gel. The gel is injected in amounts of 1 cc to 10 cc to animals for treatment of diarrhea, depending on the size of the animal.

### Example 2

A chewable tablet was prepared with the following ingredients:

| | GRAM PER TABLET |
|---|---|
| | (UNLESS OTHERWISE |
| INGREDIENT | INDICATED) |
| Kaolin | 1.0 g |
| Pectin | 0.050 g |
| Sorbitol | 1.250 g |
| Maltodextrin | 0.610 g |
| liver flavor | 0.390 g |
| microcrystalline cellulose | 0.200 g |
| Dextrose(sweetener system) | 0.055 g |
| whey protein | 0.250 g |
| Encapsulated dried | 0.6 billion colony-forming units |
| Enterococcus faecium | (0.13869 billion colony-forming units/gm of |
| Fermentation product | tablet) |
| Brewer's Yeast | 0.200 g |
| dicalcium phosphate | 0.050 g |
| stearic acid | 0.225 g |
| Magnesium stearate | 0.008 g |
| Silica | 0.038 g |

4.326 gm/tablet

As prepared, the tablets are administered to dogs and cats for the treatment of diarrhea in dosages of one or more tablets depending on the size of the animal.

The preparations according to the invention will help resolve diarrhea that may be caused by stress, antibiotic administration, change in diet, or other simple, non-life threatening forms of diarrhea in small domestic animals such as dogs and cats. The combination of ingredients is designed to help stop diarrhea in pets, sooth irritated intestines and restore the normal balance of intestinal bacteria.

Reasonable variation and modifications are possible within the scope of foregoing disclosure without departing from the spirit of the invention which is set forth in the appended claims.

What is claimed is:

1. A composition for treating diarrhea in small domestic animals such as dogs and cats comprising:
an effective amount of kaolin, pectin and at least one probiotic; and a carrier for the kaolin, pectin, and at least one probiotic.

2. A composition according to claim 1, wherein the composition is formed in a gel and the kaolin is present in the amounts of about 20-80 parts by weight.

3. A composition according to claim 2, wherein the kaolin is preferably in the range of 47-50 weight percent.

4. A composition according to claim 3, wherein the pectin is present in amounts of 0.5-10 weight percent.

5. A composition according to claim 4, wherein the pectin is present in amounts of about 1.9-2 percent by weight.

6. A composition according to claim 5, wherein the at least one probiotic is selected from the group of Aspergillus niger;Aspergillus oryzae; Bacillus lentus; Bacillus licheniformis; Bacillus pumilus; Bacillus subtilis; Bacteroides amylophilus; Bacteroides capillosus; Bacteroides ruminocola; Bacteroides suis; Bifidobacterium adolescentis; Bifidobacterium animalis; Bifidobacterium bifidum; Bifidobacterium infantis; Bifidobacterium longum; Bifidobacterium thermophilum; Lactobacillus acidophilus; Lactobacillus brevis; Lactobacillus buchneri; Lactobacillus bulgaricus; Lactobacillus casei; Lactobacillus cellobiosus; Lactobacillus curvatus; Lactobacillus delbruekii; Lactobacillusfermentum; Lactobacillus helveticus; Lactobacillus lactis; Lactobacillus plantarum; Lactobacillus euterni (probably reuterni or reuteri); Leuconostoc mesenteroides; Pediococcus acidilacticii; Pediococcus cerevisiae (damnosus); Propionibacterium freudenreichii; Propionibacterium shermanii; Saccharomyces cerevisiae; Enterococcus cremoris; Enterococcus diacetylactis; Enterococcus faecium (E. faecium); Enterococcus intermedius; Enterococcus lactis; Enterococcus thermophilus; Yeast.

7. A composition according to claim 6, wherein the at least one probiotic is E. faecium.

8. A composition according to claim 7, wherein the at least one probiotic is present in the range of about 50-400 million colony-forming units per gram.

9. A composition according to claim 8, wherein the at least one probiotic is present in the range of about 100-200 million colony-forming units per gram.

10. A composition according to claim 9 wherein the carrier comprises an edible oil in the amount of about 20-60% by weight.

11. A composition according to claim 10, wherein the range of the edible oil is 41-44% by weight.

12. A composition according to claim 10 wherein the oil is soy oil.

13. A composition according to claim 2, wherein the pectin is present in amounts of 0.5-10 weight percent.

14. A composition according to claim 2, wherein the at least one probiotic is selected from the group of Aspergillus niger;Aspergillus oryzae; Bacillus lentus; Bacillus licheniformis; Bacillus pumilus; Bacillus subtilis; Bacteroides amylophilus; Bacteroides capillosus; Bacteroides ruminocola; Bacteroides suis; Bifidobacterium adolescentis; Bifidobacterium animalis; Bifidobacterium bifidum; Bifidobacterium infantis; Bifidobacterium longum; Bifidobacterium thermophilum; Lactobacillus acidophilus; Lactobacillus brevis; Lactobacillus buchneri; Lactobacillus bulgaricus; Lactobacillus casei; Lactobacillus cellobiosus; Lactobacillus curvatus; Lactobacillus delbruekii; Lactobacillus fermentum; Lactobacillus helveticus; Lactobacillus lactis; Lactobacillus plantarum; Lactobacillus euterii (probably reuterii or reuteri); Leuconostoc mesenteroides; Pediococcus acidilacticii; Pediococcus cerevisiae (damnosus); Propionibacterium freudenreichii; Propionibacterium shermanii; Saccharomyces cerevisiae; Enterococcus cremoris; Enterococcus diacetylactis; Enterococcus faecium (E. faecium); Enterococcus intermedius; Enterococcus lactis; Enterococcus thermophilus; Yeast.

15. A composition according to claim 14, wherein the at least one probiotic is present in the range of about 50-400 million colony-forming units per gram.

16. A composition according to claim 15 wherein the at least one probiotic is present in the range of about 100-200 million colony-forming units per gram.

17. A composition according to claim 16 wherein the carrier comprises an edible oil in the amount of about 20-60% by weight.

18. A composition according to claim 17 wherein the range of the edible oil is 41-44% by weight.

19. A composition according to claim 2, wherein the at least one probiotic is E. faecium.

20. A composition according to claims 2 wherein the carrier comprises an edible oil in the amount of about 20-60% by weight.

21. A composition according to claim 1 wherein the composition is in the form of a gel consisting essentially of kaolin in the range of 47-51 weight %, pectin in the range of about 1.9-2.0 weight % and the at least one probiotic is E. faecium which is present in the range of 100-200 million colony-forming units per gram, and the carrier is soy oil and is present in the range of 41-44% by weight.

22. A composition according to claim 1 wherein the composition is formed in tablet form and kaolin is present in the amounts of about 10-50 parts by weight.

23. A composition according to claim 22 wherein the kaolin is present in the range of 20-30% by weight.

24. A composition according to claim 23, wherein the kaolin is present in about 25% by weight.

25. A composition according to claim 23 wherein pectin is present in amount of 0.5-10 weight %.

26. A composition according to claim 25 wherein the pectin is present in amounts of about 1-2% by weight.

27. A composition according to claim 23 wherein the at least one probiotic is present in the amount of about 50-400 million colony-forming units per gram.

28. A composition according to claim 27 when the carrier includes sorbitol in the range of 10-40% by weight.

29. A composition according to claim 28, wherein the carrier further includes maltodextrin in the range of 5-25% by weight.

30. A composition according to claim 22 wherein the at least one probiotic is selected from the group of Aspergillus niger; Aspergillus oryzae; Bacillus lentus; Bacillus licheniformis; Bacillus pumilus; Bacillus subtilis; Bacteroides amylophilus; Bacteroides capillosus; Bacteroides ruminocola; Bacteroides suis; Bifidobacterium adolescentis; Bifidobacterium animalis; Bifidobacterium bifidum; Bifidobacterium infantis; Bifidobacterium longum; Bifidobacterium thermophilum; Lactobacillus acidophilus; Lactobacillus brevis; Lactobacillus buchneri; Lactobacillus bulgaricus; Lactobacillus casei; Lactobacillus cellobiosus; Lactobacillus curvatus; Lactobacillus delbruekii; Lactobacillus fermentum; Lactobacillus helveticus; Lactobacillus lactis; Lactobacillus plantarum; Lactobacillus euterii (probably reuterii or reuteri); Leuconostoc mesenteroides; Pediococcus acidilacticii; Pediococcus cerevisiae (damnosus); Propionibacterium freudenreichii; Propionibacterium shermanii; Saccharomyces cerevisiae; Enterococcus cremoris; Enterococcus diacetylactis; Enterococcusfaecium (E. faecium); Enterococcus intermedius; Enterococcus lactis; Enterococcus thermophilus; Yeast.

31. A composition according to claim 30 wherein the at least one probiotic is E. faecium.

32. A composition according to claim 31 wherein the at least one probiotic is present in the amount of about 50-400 million colony-forming units per gram.

33. A composition according to claim 32 wherein the at least one probiotic is in the range of 100-200 million colony-forming units per gram.

34. A composition according to claim 33 wherein the carrier comprises sorbitol which is present in the range of about 26-32 weight %.

35. A composition according to claim 1 and further including a flavoring that is attractive to domestic animals.

36. A composition according to claims 35 wherein the flavoring is liver flavor.

37. A composition according to claim 1 wherein the composition is in tablet form and consists essentially of: about 10-50% by weight of kaolin; about 0.5-10% by weight of pectin; about 50-400 million colony-forming units per gram of E. faecium; about 10-40 weight % of sorbitol; about 5-25 weight % of maltodextrin; and liver flavor.

38. A composition according to claim 1 wherein the composition is in tablet form and consists essentially of: about 25% by weight kaolin; about 1.25% by weight pectin; about 100-200 million colony-forming units per gram of E. faecium; about 26-32 weight % of sorbitol; about 12-15 weight % of maltodextrin; and liver flavor.

39. A composition according to claim 38 wherein the ratio of kaolin to pectin is in the range of about 35:1 to 15:1.

40. A composition according to claim 38 wherein the ratio of kaolin to pectin is in the range of about 25:1.

41. A composition according to claim 1 wherein the composition is in the form of a gel consisting essentially of kaolin in the range of 20-80 weight %, pectin in the range of about 0.5-10 weight % and the at least one probiotic is E. faecium which is present in the range of 50-400 million colony-forming units per gram, and the carrier is soy oil and is present in the range of 20-60% by weight.

42. A method for treating diarrhea in small domestic animals such as dogs and cats comprising the steps of orally administering an effective dosage of the composition of claim 1 to the animal.

43. A method of treating diarrhea in small domestic animals such as dogs and cats comprising the steps of orally administering an effective dosage of the composition of claim 2 to the animal by injecting the dosage in gel form into the animal's mouth with a syringe.

44. A method of treating diarrhea in small domestic animals such as dogs and cats comprising the steps of orally administering an effective dosage of the composition of claim 22 to the animal by mixing the effective dosage with the animals food or otherwise orally administering the composition to the animal.
